(19) United States
(12) Patent Application Publication
(10) Pub. No.: US 2005/0217925 A1
(43) Pub. Date: Oct. 6, 2005

(54) Hearing protection device

(76) Inventor: Randy E. Benway, Horicon, WI (US)

Correspondence Address: RYAN KROMHOLZ & MANION, S.C., POST OFFICE BOX 26618, MILWAUKEE, WI, 53226, US

(21) Appl. No.: 10/816,458

(22) Filed: Apr. 1, 2004

(51) Int. Cl. H04R 25/00; H04R 25/02; A61B 7/02; A42B 1/06

(52) U.S. Cl. 181/130; 181/135; 181/129

(57) ABSTRACT

An ear hearing protection unit is preferably coupled with a conventional hat, such as a baseball cap, and preferred routing channels extend proximal to an ear of a wearer of the hat.

### BACKGROUND OF THE INVENTION

This invention relates to ear-worn hearing protection, particularly suited for loud noise environments such as workplaces or firearms ranges.

Hearing protectors fall generally into three categories, including protectors that cap the entrance to the ear canal; protectors which enter the ear canal and seal the ear canal prior to the bend in the ear canal (usually referred to simply as semi-insert devices); and protectors that enter the ear canal and take the bend in the ear canal (sometimes referred to as banded earplugs). Semi-insert hearing protectors generally protect similarly to earplugs, but usually to a lesser level. Semi-insert hearing protectors are also referred to as semi-aural hearing protectors. Semi-insert hearing protectors which enter the ear canal to a greater degree offer better protection but are somewhat less comfortable than those which simply cap the ear. Products which cap the ear have some of the attributes of both earplugs and earmuffs. Typically, they are used for intermittent noise exposures where lighter weight and improved low frequency attenuation are desirable.

### SUMMARY OF THE INVENTION

A hearing protector is disclosed that amplifies desirable sounds, such as voices, but suppresses loud sounds such as shotgun discharges.

Electronic hearing protection exists for the shooting industry. Ear muff styles are worn over the ears during shooting. External microphones sample sound and send the signals to circuitry inside that amplifies the signal and sends it to internal speakers. Volume controls are typically on each earpiece. Muffs are very effective, inexpensive, but large. Size is the biggest problem for the shot-gunner as many times the gunstock contacts the muffs upon mounting the shotgun to the shoulder.

Separate electronic plugs are also available. They can be of the type that are inserted into ear, or clip behind the ear. They too have the same features such as volume controls, as the muff style. Because of their small size, cost of this type ear protection is considerable.

The present invention is a design where the microphone, speaker, and other circuitry is packaged in a single housing that could be affixed to the underside of a cap brim, at a greatly reduced cost to the prior art.

Tubing from the module is routed through the hat, exiting the sides, where it is connected to earphones, much like a stethoscope. Mono or stereo models can be provided.

The present invention comprises a single ear hearing protection unit, coupled to a tee-fitting, further coupled to 1/16″ I.D. tubing, and 90° fittings. The single ear hearing protection unit can be coupled with a conventional hat, such as a baseball cap. A single tube may be routed from the single ear hearing protection unit rearward to a tee fitting. A pair of tubes is coupled to the tee fitting toward each ear. 90° fittings are then coupled to each of the tubes that are coupled to the tee-fitting, which are in turn coupled to additional tubes and finally to ear plugs, which the user places in the ears during use.

### BRIEF DESCRIPTION OF THE DRAWINGS

FIG. 1 is a top perspective view of a preferred embodiment of a hearing protection device of the present invention.

FIG. 2 is a bottom perspective view of a preferred embodiment of a hearing protection device of the present invention.

FIG. 3 is a top perspective view of a second preferred embodiment of a hearing protection device of the present invention.

FIG. 3a is a side view of an alternate embodiment of a microphone/speaker arrangement of the present invention.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structure. While the preferred embodiment has been described, the details may be changed without departing from the invention, which is defined by the claims.

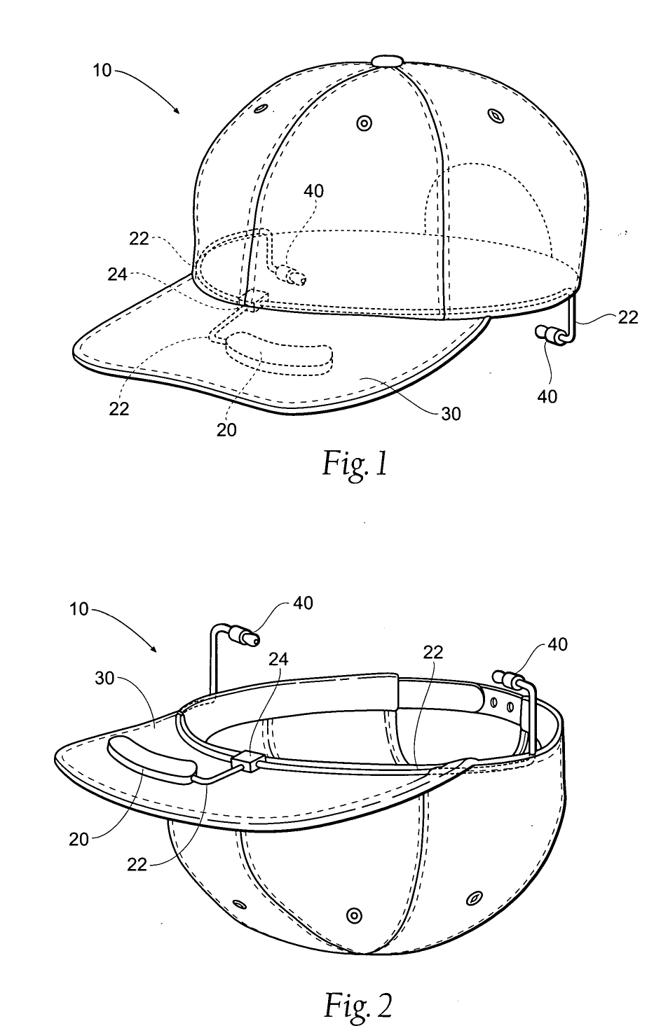

Referring to FIG. 1, a top perspective view of a preferred embodiment of a hearing protection device 10 of the present invention is shown.

In a preferred embodiment, a microphone, speaker, and other circuitry 20 is packaged in a single housing that is preferably affixed to an underside of a brim of a cap 30. This conceals the hearing protection device 10 in a preferred embodiment.

Tubing from the microphone, speaker, and other circuitry 20 is routed through the cap 30, exiting the sides, where it is connected to earphones 40. Mono or stereo models can be provided.

One advantage of the present invention is that typical in-the-ear and behind-the-ear units that require small ‘hearing aid’ type batteries can be used. These are typically zinc-air batteries that start discharging as soon as they are installed. Alternatively, a behind-the-ear type module can be used, providing approximately two weeks per battery, in use about 1 hour per week. A larger module can also be provided, using typical watch batteries (or larger) for much longer periods of intermittent use.

The present invention comprises a single ear hearing protection unit 20, coupled to tubing 22, next to a tee-fitting 24, further coupled to more tubing 22, such as 1/16″ I.D. tubing, and next either the tubing is bent or coupled with 90° fittings (not shown) which then direct the tubing down from the cap 30 toward the ear of a user. Last, ear plugs 40 are placed in the ears during use.

The single ear hearing protection unit is preferably coupled with a conventional hat, such as a baseball cap.

Referring to FIG. 2, a bottom perspective view of a preferred embodiment of the hearing protection device 10 of the present invention is shown.

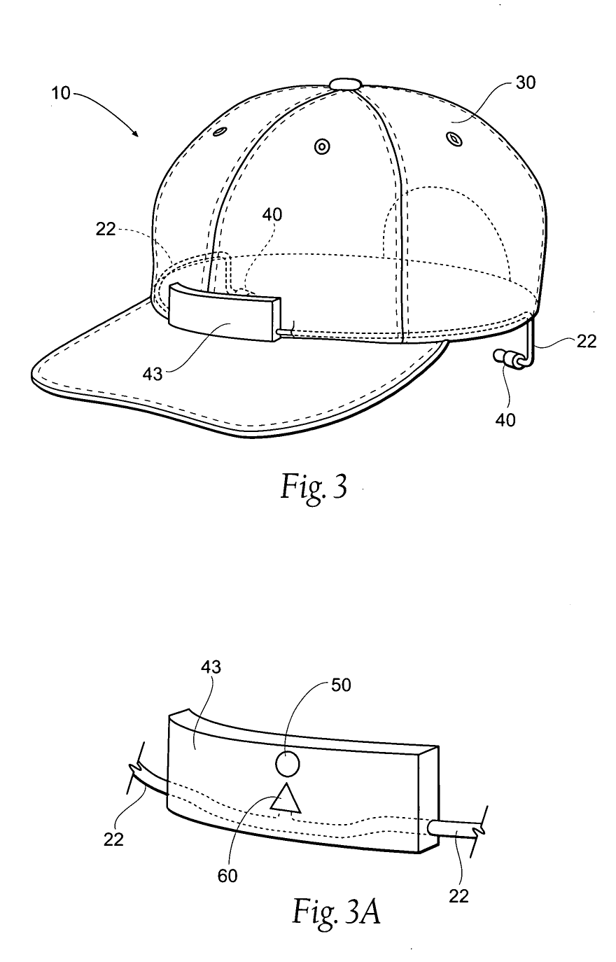

Referring now to FIG. 3 a top perspective view of a second preferred embodiment of a hearing protection device 10 of the present invention is shown. In this embodiment, a microphone/speaker assembly 43 is coupled on the outside of the cap 30, preferably on the front of the cap, where designs or slogans may be emblazoned on the assembly 43. In this arrangement, tubing 22 can either be routed through the cap 30, as shown, and then in the area proximal to the ear, or routed about the hat and routed rearward (not shown).

As will be appreciated, the microphone/speaker assembly 43 could also be coupled to different portions of the cap 30, such as the top of the brim, or the back, in accordance with manufacturer preference.

Referring now to FIG. 3a, a side view of the microphone/speaker assembly 43 is shown. As is there shown, a microphone 50 is provided coupled internally to internal 60, again further coupled downstream to the tubing 22 and ultimately into earpiece 40.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

1. A hearing protection device comprising:
a microphone coupled to a speaker;
tubing coupled between the speaker and at least one ear of a user;
said microphone and speaker and said tubing carried by a hat.

2. A hearing protection device according to claim 1, the device further comprising a first length of tubing coupled to said microphone and said speaker, said first length of tubing coupled to a tee, and a second length of tubing coupled to an ear piece.

3. A hearing protection device according to claim 1, said device further comprising a mono hearing unit, said mono hearing unit providing identical signals to a right ear and a left ear.

4. A hearing protection device according to claim 1, said device further comprising a stereo hearing unit, said stereo hearing unit providing different signals to a right ear and a left ear.

5. A hearing protection device according to claim 1, wherein said tubing is coupled between said speaker and said at least one ear of a user above a hat brim.

6. A hearing protection device according to claim 1, wherein said tubing is coupled between said speaker and said at least one ear of a user below a hat brim.

7. A hearing protection device according to claim 1, wherein said microphone and speaker are carried by said hat underneath a brim of said hat.

8. A microphone coupled to a speaker;
wiring coupled between the speaker and the microphone,
said microphone and speaker and said wiring carried by a hat;
said speaker positioned proximal to at least one ear of a wearer of said hat when said hat is worn;
said speaker suppressing loud sounds such as shotgun discharges.
